(19) United States
(12) Patent Application Publication
(10) Pub. No.: US 2010/0226862 A1
(43) Pub. Date: Sep. 9, 2010

(54) MICROCAPSULE AND METHOD FOR MAGNETIC RESONANCE IMAGING TO LOCALIZE BLOOD

(76) Inventor: Sebastian Schmidt, Weisendorf (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/717,303

(22) Filed: Mar. 4, 2010

(30) Foreign Application Priority Data
Mar. 4, 2009 (DE) 10 2009 011 607.9

(51) Int. Cl. A61B 5/055; A61K 9/50

(52) U.S. Cl. 424/9.32; 424/9.3; 424/9.37; 424/9.36; 424/490; 977/773

(57) ABSTRACT

Microcapsule for injection into the bloodstream of a patient before a magnetic resonance acquisition pertaining to the bloodstream, include a magnetic resonance marking substance (in particular with at least one isotope associated with a specific resonant frequency) inside the microcapsule and an outer membrane that is impermeable to the marking substance. The microcapsule is overall biodegradable.

### BACKGROUND OF THE INVENTION

1. Field of the Invention

The present invention concerns a microcapsule for injection into the bloodstream of a patient before a magnetic resonance acquisition pertaining to the bloodstream, as well as a method to acquire magnetic resonance image data enabling a localization of blood in the body of a patient using such microcapsules.

2. Description of the Prior Art

It is frequently necessary in medicine to localize hemorrhages, especially in the gastrointestinal tract of a patient exhibiting chronic blood loss. Although decomposition products of the blood can be detected in stool, the location of the bleeding within the gastrointestinal tract is unknown.

In order to be able to localize such hemorrhages, an endoscopic examination for example, can be implemented. The upper gastrointestinal tract (esophagus and stomach) and the large intestine can be shown well via an endoscope, but smaller hemorrhages and hemorrhages that occur only irregularly are often poorly visible. Capsule endoscopy can be conducted in the small intestine, wherein a capsule with an image acquisition device is swallowed that then acquires (for example) one to two images per second and transmits these wirelessly to a receiver located outside of the body. Here the poor sensitivity of the method—since the capsule is transported passively and a hemorrhage thus can evade the field of view of the image acquisition device—is disadvantageous. Although this problem is avoided by a technique known as double balloon endoscopy, this technique is very invasive and uncomfortable for the patient.

An angiographic procedure is known that selectively shows the branch of the arteria mesenterica by means of an inserted catheter and maps the drainage of a contrast agent into the gut lumen by means of digital subtraction angiography. The sensitivity of x-ray-based digital subtraction angiography, however, is often insufficient if only small amounts of contrast agent cross over into the intestine. Again, a very invasive procedure is additionally necessary.

### SUMMARY OF THE INVENTION

An object of the present invention is to provide a method as well as associated apparatus that non-invasively enable localization of hemorrhages with high sensitivity, spatial resolution and additional anatomical information, while imposing only a low stress on the patient.

According to the invention a microcapsule is provided for injection into the bloodstream of a patient before a magnetic resonance acquisition pertaining to the bloodstream, this microcapsule embodying a magnetic resonance marking substance (in particular with at least one isotope associated with a specific resonant frequency) arranged inside the microcapsule, and an outer membrane that is impermeable to the marking substance, and the overall microcapsule is biodegradable.

The microcapsules can be considered as a type of (artificial or natural) corpuscle marked for the magnetic resonance image acquisition that can be administered to a patient before a magnetic resonance image acquisition. If a hemorrhage exists within the body, for example in the intestine, the corpuscles (thus the microcapsules) that are marked in this way accumulate there and can be seen there in the resulting magnetic resonance image data set. Magnetic resonance offers not only the advantage that it can react extremely sensitively to smaller amounts of the marking substance, but also that a high spatial resolution is possible. Anatomical exposures can additionally be made with the same image acquisition device so that the relationship of the detected hemorrhage to the anatomy of the patient can be established extremely easily. The microcapsules ideally exhibit dimensions similar to the red corpuscles present anyway in the blood; they can exhibit a maximum length of 1-20 μm, in particular of 5-10 μm.

The microcapsules used as marked corpuscles structurally exhibit a membrane that encloses an internal space in the magnetic resonance marking substance is contained. The membrane is impermeable to the marking substance, but biodegradable. The marking substance thus exists in the microcapsule in a high concentration but is dispensed into the blood only very slowly with the degradation of the microcapsule over days to weeks, which increases the compatibility.

If the microcapsules are consequently used (for example by intravenous injection of microcapsules into the bloodstream of the patient) before the magnetic resonance acquisition (which then naturally ensues in an acquisition technique matched to the marking substance), a non-invasive method (described below in detail) for certain detection of hemorrhages is provided, in particular in the gastrointestinal tract of the patient.

The magnetic resonance marking substance (the isotope) should be selected so that natural occurrence thereof in the human body is extremely slight in order to be able to have a weak background and a clear signal. There are essentially two different types of marking substances. Isotopes with a net spin and a specific resonance frequency are detected simply and with certainty by excitation and measurement at their respective resonant frequency. Marking substances are also known in which a negative contrast results that is clearly visible under specific acquisition techniques, in particular T2-weighted or T2*-weighted sequences. Some examples of marking substances are as follows.

The marking substance can be a fluorine compound, in particular a perfluorocarbon. The low occurrence of fluorine in the human body is advantageous, so only a minimal background signal results. Perfluorocarbons are known as blood substitutes (artificial blood) and are very compatible. Many magnetic resonance image acquisition devices are additionally already designed to measure at the corresponding resonant frequency.

Another possibility is the use of compounds of high molecule weight marked with isotopes exhibiting a net spin (in particular 13-C or 15-O), in particular starch compounds and/or sugar compounds. An example is hydroxyethyl starch marked with 13-C. Such polysaccharides likewise prove to have excellent compatibility.

Among marking substances that generate a negative contrast, for example, iron oxide nanoparticles or gadolinium chelates can be used. Such markers produce a signal cancellation that is detected well in T2-weighted or T2*-weighted image data sets.

Alternatively, the signal amplification in the T1-weighted image can be shown dependent on the concentration of the marking substance.

It is generally important that the marking substance cannot penetrate the microcapsule membrane, which is true for all of the previously cited substances.

As already mentioned, the microcapsules employed as marked corpuscles can be natural corpuscles (marked, red corpuscles) but can also be artificial corpuscles. For example, the microcapsule can be an erythrocyte ghost developed from a red blood cell. Such erythrocyte ghosts are known in principle and are used in basic physiological research, for example. To produce such ghosts, erythrocytes (thus red blood cells) are washed and lysed in a hypotonic solution (for example in 4 mM magnesium sulfate). Because the corpuscles are exposed to a solution that contains less salt than the blood, they significantly expand so that their membranes become permeable and hemoglobin can exit. After this, the erythrocytes are reconstituted in an isotonic solution, for example in 0.9 NaCl with Tris buffer. In this reconstitution solution, the marking substance is now added, that is incorporated inside the erythrocyte ghost because the original form of the red corpuscles is reestablished in the isotonic solution, such that the membrane is again impermeable. Finally, the developed erythrocyte ghosts are washed again. If the microcapsules are produced from red corpuscles as described above, an excellent compatibility is already provided if the blood cells used originate from the patient to be imaged and/or from his or her blood bank. If donated erythrocytes are used (for example from erythrocyte concentrates from blood banks), blood group compatibility must be insured. However, it can also be possible for at least one surface marker of the erythrocyte ghost, in particular surface markers associated with the blood group, to be destroyed or inactive. This can be achieved by adding denaturing substances (for example aldehyde or ethanol) during the preparation, that reduce the immunogenicity of the erythrocytes. “Universally marked corpuscles” can ultimately be produced in this way.

The microcapsules can also be produced artificially. The microcapsule can be a liposome with an internal space in which the marking substance is located. In such an “artificial corpuscle” embodiment it must be ensured that these have approximately the same size as natural corpuscles. Liposomes are known and are a specific arrangement of surface-active molecules (in particular of lipids) in a fluid. The surface-active molecules arrange themselves with the hydrophilic side outward, so the lipophilic remainder forms a membrane. The liposomes thereby produce as a structure held together by molecular forces that frequently exhibits a spherical shape. The marking substance can then be arranged inside. Another example of artificial microcapsules is, for example, alginate microcapsules.

In addition to the microcapsule according to the invention, the present invention also concerns a method to acquire magnetic resonance image data enabling localization of blood in the body of a patient, wherein at least one magnetic resonance image data set of a region of interest is acquired after an injection of a predetermined number of microcapsules into the bloodstream of the body, using an acquisition technique that causes the marking substance to be visible in the resulting image.

Microcapsules (that can also be viewed as marked corpuscles) are thus prepared in order to become carriers of a marking substance. These can then be introduced into the bloodstream of a patient, in particular by intravenous injection. According to the invention, a microcapsule image data set is acquired with an acquisition technique matched to the marking substance, from which microcapsule image data set it is clear where blood is present in the field of view that encompasses the region of interest. In this way it is possible with a highly sensitive image acquisition technique with high spatial resolution to localize hemorrhages (for example) that manifest as accumulations of the microcapsules (consequently the marking substance) at locations at which no blood vessels are present. Hemorrhages (in particular in the gastrointestinal tract) can thus be discovered and localized non-invasively and safely by the subsequent evaluation of the image data which can, for example, be conducted by a physician or even automatically. Natural or artificial corpuscles marked according to the invention (the microcapsules) are thus used to detect hemorrhages (in particular gastrointestinal hemorrhages) by means of magnetic resonance. The microcapsule image data sets that are created show the crossing of the microcapsules into the intestine and enable (by their high resolution) simple locating of the hemorrhage point and a clear association with anatomical structures, in particular if the anatomical structures are detectable in the microcapsule image data set itself or also in an additionally acquired anatomy image data set.

At least one anatomy image data set showing the anatomy in the region of interest can be acquired promptly, in particular with the patient's body not being moved in comparison to the microcapsule image data set. Such an anatomy image data set proves to be particularly useful if the marking substance includes an isotope with a specific resonance frequency that otherwise occurs rarely in the body, since anatomical structures are for the most part poorly recognizable or not detectable at all in these images. By the additional acquisition of an anatomy image data set, a comparison is available that simplifies the localization with regard to anatomical structures. A fusion image data set can advantageously be determined by fusion of the microcapsule image data set and the anatomy image data set. An image that can be shown at a presentation device then results, in which both the anatomical structures and the occurrence of blood (in particular hemorrhages) are easily detectable.

Different types of hemorrhages also manifest themselves differently in the microcapsule image data sets. For example, if an acute, stronger bleeding is present, the magnetic resonance measurement yields a particularly high contrast if it is implemented during the first arterial passage of the microcapsules. However, smaller or irregular hemorrhages can also occur in which the marked microcapsules accumulate in the intestine, for example, and can be clearly measured after some time. Accordingly, multiple microcapsule image data sets are acquired at different points in time, in particular at intervals of a few minutes. An example of a measurement protocol, for example, is one that allows a microcapsule image data set to be acquired as a reference image data set immediately before the injection of the microcapsules. The injection occurs immediately after this, whereupon a first microcapsule image data set is acquired after 25 seconds, for instance. This essentially corresponds to the time that microcapsules intravenously injected into the arm require to reach the corresponding arteries in the gastrointestinal tract. Then an additional microcapsule image can be acquired after approximately 5 minutes have elapsed, for example.

Comparisons between different microcapsule image data sets are also conceivable. At least one subtraction image data set can be determined by subtraction of two microcapsule image data sets. This is particularly advantageous when a reference microcapsule image data set is acquired at a point in time at which the microcapsules have not yet reached the region of interest. The background can then be advantageously excluded from consideration.

It need only be noted that, in the case of fusion or other image processing operations, in particular given acquisition of the gastrointestinal tract, it must be taken into account whether a movement correction is necessary. Techniques for movement correction are generally known, and need not be presented in detail herein.

As mentioned, a negative contrast results given a marking with iron oxide nanoparticles or even gadolinium chelates. In order to be able to show these particularly well, it can be provided that the acquisition technique comprises a T2-weighted or T2*-weighted sequence given the use of iron oxide nanoparticles or a gadolinium chelate as a marking substance. The negative contrast that is created is shown well.

In particular given the use of iron oxide nanoparticles or a gadolinium chelate as a marking substance in a region of interest of the patient that comprises at least a portion of the gastrointestinal tract before acquisition of the microcapsule image data set, an aqueous solution (in particular water with methyl cellulose) can be particularly advantageously administered orally. In this way the intestine can be shown better in the image since the intestine contents then show up with very high signal (in particular in the T2*-weighted image) so that the signal cancellation due to iron oxide or gadolinium chelate is very recognizable in the event that the crossover of the marked corpuscles (thus the microcapsules) into the intestine occurs. The methyl cellulose (cited as an example) thereby acts as a thickening agent which prevents the orally administered aqueous solution from leaving the intestine too quickly.

### BRIEF DESCRIPTION OF THE DRAWINGS

FIG. 1 shows a microcapsule of a first embodiment according to the invention.

FIG. 2 shows a microcapsule according to the invention in a second embodiment.

FIG. 3 is a flowchart of an embodiment of a method for localization hemorrhages in the gastrointestinal tract in accordance with the invention.

### DESCRIPTION OF THE PREFERRED EMBODIMENTS

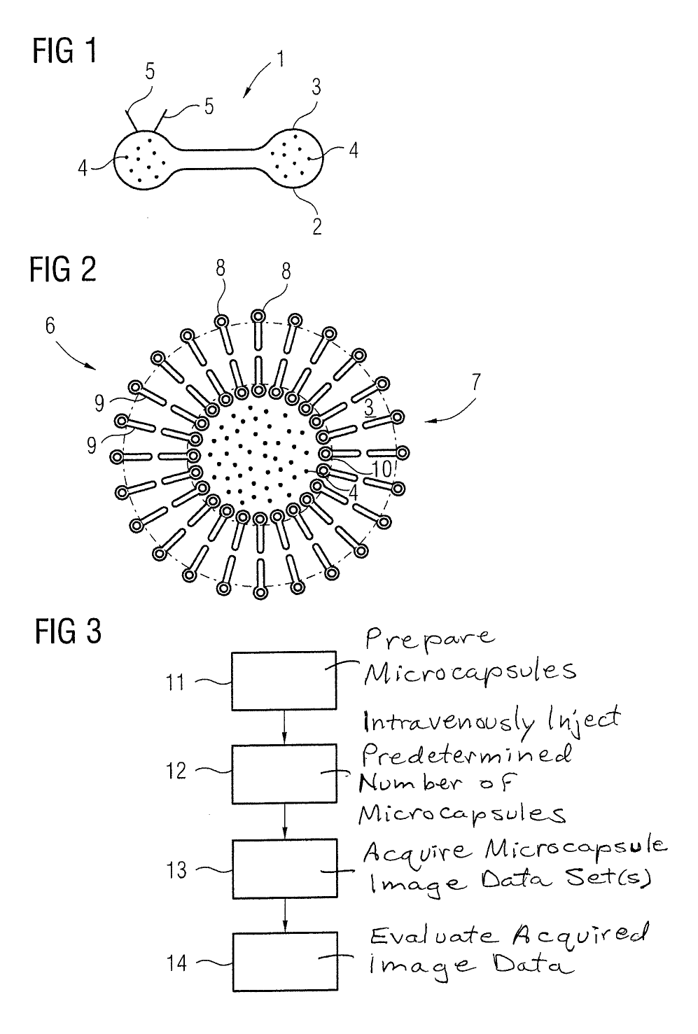

FIG. 1 shows as a first exemplary embodiment a microcapsule 1 according to the invention (that can also be designated as a marked corpuscle), an erythrocyte ghost 2 developed from a red blood cell. The microcapsule 1 comprises the natural corpuscle membrane 3 and a magnetic resonance marking substance 4 arranged inside that exists in a high concentration in the microcapsule 1 and for which the membrane 3 is impermeable. Nevertheless, a good biodegradability is ensured since the erythrocyte ghost 2 is based on a natural red blood cell.

The corpuscles that are used can originate from the patient to be examined, or also from a blood bank. In the present case, the surface markers associated with the blood group (schematically indicated at 5) are destroyed, which means that the immunogenicity of the microcapsules 2 is reduced. This can be achieved if denaturing substances (for example aldehydes or ethanol) are added during the preparation.

In order to produce such erythrocyte ghost 2, erythrocytes (red blood cells) are initially washed in order to then be lysed with a hypotonic solution. The membrane 3 is reestablished in an isotonic solution in which the marking substance 4 is also contained, such that the marking substance 4 is incorporated into the erythrocyte ghost that is created. The erythrocyte ghost can then be washed again.

Multiple possibilities can be taken into consideration for a marking substance 3, for example a fluorine compound (in particular a perfluorocarbon) as explained above, or compounds (for example hydroxyethyl starch) of high molecular weight marked with isotopes (for example 13-C) exhibiting a net spin, from the series of marking substances possessing isotopes with particular resonance frequencies; however, a marking substance 4 generating a negative contrast can also be used, for example iron oxide particles or a gadolinium chelate.

These marking substances can also be used in the second exemplary embodiment of a microcapsule according to the invention that is shown in FIG. 2. This is a liposome 7 which has formed from lipid molecules that comprise a hydrophilic portion 8 and a hydrophobic portion 9. The hydrophilic portion 8 points outward or inward and thus shields the hydrophobic portions from the fluid. The hydrophobic (and this lipophilic) remainder in this case forms the membrane 3. A marking substance 4 is thereby in turn arranged in an internal space 10.

The liposome 7 is spherical and presently exhibits a diameter of 10 μm, thus is approximately as large as a red blood cell, such that here it can be discussed as an artificial corpuscle. The microcapsules 1, 6 described in FIG. 1 or, respectively, FIG. 2 can advantageously be used in order to be able to localize a hemorrhage in the gastrointestinal tract of a patient. For example, this can proceed as shown in FIG. 3.

Microcapsules (thus marked, natural or artificial corpuscles) are thereby prepared in Step 11. A predetermined number of these microcapsules is intravenously injected into a patient in Step 12. The predetermined number can thereby be, for example, 10⁸-10¹⁰ microcapsules, which corresponds approximately to the amount of erythrocytes in 0.1-10 ml of blood.

One or more microcapsule image data sets are then acquired in Step 13 using an acquisition technique showing the marking substance, in particular after the microcapsules have passed the gastrointestinal tract into the arteries there. If a hemorrhage exists, the microcapsules (and thus the marking substance) consequently accumulate in the intestine so that a clear image signal indicating the hemorrhage with high spatial resolution is created since the acquisition technique is matched to the marking substance. While a suitable image signal might already occur after the first passage of the microcapsules in the gastrointestinal tract given acute, strong bleeds, given less strong bleeds a certain time (for example a few minutes) can be waited under sufficient microcapsules have accumulated in the intestine.

If the marking substance comprises specific isotopes with a resonance frequency (for example fluorine), this resonance frequency is excited. For example, magnetic resonance antennas are known for this that can be adjusted to different resonant frequencies (for example also to that of fluorine) so that such exposures can be produced. In this context it is reasonable in principle to also generate an anatomy image data set via the typical proton imaging so that the anatomy can be correlated with the locations that can be determined via the microcapsule image data set. This anatomy image data set should be acquired promptly, in particular if the patient has not yet moved in comparison to the acquisition of the microcapsule image data set. In particular, it is possible to generate a fusion image data set via fusion of the microcapsule image data set and the anatomy image data set, which fusion image data set then contains the anatomical information and the blood-related information.

If a marking substance is used that causes a negative contrast (for example iron oxide nanoparticles), an aqueous solution (for example water with methyl cellulose) is orally administered to the patient before the examination, whereupon a T2*-weighted sequence is used as an acquisition technique. The intestine itself thereby shows up with very high signal, such that the signal cancellation by the iron oxide is very well recognizable in the event that a crossover of the marked microcapsules into the intestine occurs (thus a hemorrhage is present).

If multiple microcapsule image data sets are acquired, it is thereby also possible to compare these, for example to form subtraction image data sets. This is particularly advantageous if a reference microcapsule image data set was generated (for example shortly before the injection of the microcapsules) in which no signal (or also no non-signal) caused by the microcapsules exists. The background that is possibly present can be eliminated in this way.

For example, it can be provided that such a reference microcapsule image data set is acquired immediately before the administration of the microcapsules; the injection of the microcapsules then occurs, an additional microcapsule image data set is acquired after approximately 25 more seconds, and a third microcapsule image data set is acquired after five minutes so that the temporal development can also be observed.

The acquired image data are evaluated in Step 14. For example, for this it can be provided that an image data set (for example a microcapsule image data set itself, a subtraction image data set or a fusion image data set) is possibly displayed in parallel with the display of an anatomy image data set on the display device, such that a physician can assess the presence of hemorrhages. Naturally, an automatic evaluation is also possible in principle.

It may be necessary—for example due to the intestinal movement—to conduct a movement correction in the comparison of different image data sets.

Although modifications and changes may be suggested by those skilled in the art, it is the intention of the inventors to embody within the patent warranted hereon all changes and modifications as reasonably and properly come within the scope of their contribution to the art.

I claim as my invention:

1. A microcapsule comprising:
a magnetic resonance marking substance comprising at least one isotope associated with a predetermined resonant frequency;
an outer membrane in which said marking substance is contained, said outer membrane being impermeable to said marking substance;
said outer membrane with said marking substance therein having a size configured for injection of said outer membrane with said marking substance therein into the blood stream of a patient; and
said outer membrane and said marking substance being biodegradable.

2. A microcapsule as claimed in claim 1 wherein said outer membrane with said marking substance therein is an erythrocyte ghost developed from a red blood cell.

3. A microcapsule as claimed in claim 2 wherein said erythrocyte ghost is developed from a corpuscle originating from a source selected from the group consisting of the patient and a blood bank.

4. A microcapsule as claimed in claim 2 wherein said erythrocyte ghost comprises at least one surface marker associated with a blood group, that is destroyed or inactive.

5. A microcapsule as claimed in claim 1 wherein said outer membrane comprises an internal space in which said marking substance is contained.

6. A microcapsule as claimed in claim 1 wherein said outer membrane with said marking substance therein has a maximum dimension in a range between 1 and 20 μm.

7. A microcapsule as claimed in claim 6 wherein said outer membrane with said marking substance therein has a maximum dimension in a range between 5 and 10 μm.

8. A microcapsule as claimed in claim 1 wherein said marking substance is a fluorine compound.

9. A microcapsule as claimed in claim 8 wherein said marking substance is a perfluorcarbon.

10. A microcapsule as claimed in claim 1 wherein said marking substance comprises iron oxide nanoparticles.

11. A microcapsule as claimed in claim 1 wherein said marking substance comprises gadolinium chelate.

12. A microcapsule as claimed in claim 1 wherein said marking substance comprises a compound having a high molecular weight marked with isotopes exhibiting a net spin.

13. A microcapsule as claimed in claim 12 wherein said isotopes are selected from the group consisting of 13-C and 15-O.

14. A microcapsule as claimed in claim 12 wherein said compound of high molecular weight is selected from the group consisting of starch compounds and sugar compounds.

15. A method to acquire magnetic resonance image data representing localization of blood in the body of a patient, comprising the steps of:
forming biodegradable microcapsules comprising a magnetic resonance marking substance, comprising at least one isotope associated with a predetermined resonant frequency, within an outer membrane;
injecting a predetermined number of said microcapsules into the blood stream of a patient; and
acquiring magnetic resonance image data from the patient after injection of the microcapsules into the blood stream of the patient, using a magnetic resonance data acquisition technique that visibly distinguishes said marking substance.

16. A method as claimed in claim 15 comprising acquiring a microcapsule image data set from a region of interest of the patient, in which said marking substance is visibly distinguishable, and additionally acquiring an anatomical image data set of the region of interest with the body of the patient being unmoved between acquisition of said microcapsule image data set and acquisition of said anatomical image data set.

17. A method as claimed in claim 16 comprising forming a fusion image data set from said microcapsule image data set and said anatomical image data set.

18. A method as claimed in claim 15 comprising acquiring a plurality of microcapsule image data sets at different points in time after injecting said microcapsules into the blood stream of the patient.

19. A method as claimed in claim 18 comprising forming a difference image by subtracting two of said plurality of microcapsules image data sets from one another.

20. A method as claimed in claim 15 comprising selecting said marking substance from the group consisting of iron oxide nanoparticles and gadolinium chelate, and acquiring said magnetic resonance image data set using a T2*-weighted data acquisition sequence as said data acquisition technique.

21. A method as claimed in claim 15 comprising selecting said marking substance from the group consisting of iron oxide nanoparticles and gadolinium chelate, and orally administering an aqueous solution of water and methyl cellulose to the patient before acquiring said magnetic resonance image data set, and acquiring said magnetic resonance image data set from a region of the patient comprising at least a portion of the gastrointestinal tract of the patient.

22. A method as claimed in claim 15 comprising displaying said magnetic resonance image data set at a display device.

23. A method as claimed in claim 15 comprising selecting said predetermined number of said microcapsules injected into the blood stream of the patient in a range between 100 million microcapsules and 10 billion microcapsules.
